(19) United States
(12) Patent Application Publication
(10) Pub. No.: US 2006/0160902 A1
(43) Pub. Date: Jul. 20, 2006

(54) Histone deacetylase inhibitors

(76) Inventors: Norbert L. Wiech, Phoenix, MD (US); Hsuan-Yin Lan-Hargest, Fallston, MD (US)

Correspondence Address: STEPTOE & JOHNSON LLP, 1330 CONNECTICUT AVENUE, N.W., WASHINGTON, DC, 20036, US

(21) Appl. No.: 11/268,546

(22) Filed: Nov. 8, 2005

Related U.S. Application Data
(60) Provisional application No. 60/625,573, filed on Nov. 8, 2004.

(51) Int. Cl. A61K 31/44; A61K 31/19

(52) U.S. Cl. 514/575; 514/357

(57) ABSTRACT

Hormone refractory metastatic disease can be treated with an oxyamide-containing compound through the inhibition of HDAC1 or HDAC2.

### CLAIM OF PRIORITY

This application claims priority under 35 U.S.C. 119(e) to U.S. Provisional Patent Application Ser. No. 60/625,573 filed Nov. 8, 2004, the entire contents of which is incorporated by reference.

### TECHNICAL FIELD

This invention relates to inhibitors of specific histone deacetylases.

### BACKGROUND

Regulation of gene expression through the inhibition of the nuclear enzyme histone deacetylase (HDAC) is one of several possible regulatory mechanisms whereby chromatin activity can be affected. The dynamic homeostasis of the nuclear acetylation of histones can be regulated by the opposing activity of the enzymes histone acetyl transferase (HAT) and histone deacetylase (HDAC). Transcriptionally silent chromatin can be characterized by nucleosomes with low levels of acetylated histones. Acetylation of histones reduces its positive charge, thereby expanding the structure of the nucleosome and facilitating the interaction of transcription factors to the DNA. Removal of the acetyl group restores the positive charge condensing the structure of the nucleosome. Acetylation of histone-DNA activates transcription of DNA's message, an enhancement of gene expression. Histone deacetylase (HDACs) can reverse the process and can serve to repress gene expression. See, for example, Grunstein, Nature 389, 349-352 (1997); Pazin et al., Cell 89, 325-328 (1997); Wade et al., Trends Biochem. Sci. 22, 128-132 (1997); and Wolffe, Science 272, 371-372 (1996).

Grozinger et al., Proc. Natl. Acad. Sci. USA, 96: 4868-4873 (1999), divides HDACs into two classes, the first represented by yeast Rpd3-like proteins, and the second represented by yeast Hda1-like proteins. This reference assigns human HDAC1, HDAC2, and HDAC3 proteins as members of a first class of HDACs, and assigns HDAC4, HDAC5, and HDAC6, as members of a second class of HDACs. HDAC7 (Kao et al., Genes & Dev., 14: 55-66 (2000), HDAC9 and HDAC10 (Ruijter et al., Biochem J., 370:737-49 (2003)) are more recent members of the second class of HDACs. HDAC8 is another new member of the first class of HDACs (Van den Wyngaert, FEBS, 478: 77-83 (2000)).

### SUMMARY

Histone deacetylase is a metallo-enzyme with zinc at the active site. Compounds having a zinc-binding moiety, such as, for example, a hydroxamic acid group, can inhibit a histone deacetylase. Certain histone deacetylase inhibitors can stabilize the acetylation of p53 leading to increases in p21 levels and Bax levels in the cell. Alternatively, the histone deacetylase inhibitors can increase p21 levels in a cell in a HDAC1 dependent but p53 independent manner. Histone deacetylase inhibitors can specifically inhibit the histone deacetylase activity of HDAC1 and/or HDAC2. Accordingly, inhibition of a specific histone deacetylase can provide an alternate route for treating cancer.

In one aspect, a method of inhibiting HDAC2 in a cell includes contacting the cell with an amount of a hydroxamic acid compound effective to inhibit deacetylation activity of HDAC2. In another aspect, a method of inhibiting HDAC1 in a cell includes contacting the cell with an amount of a hydroxamic acid compound effective to inhibit deacetylation activity of HDAC 1. The hydroxamic acid compound can be of formula (I), or a pharmaceutically acceptable salt thereof. In one embodiment, the compound further increases the levels of p21 in the cell. In another embodiment, the compound further induces cell cycle arrest in the cell. In certain circumstances, the cell can be contacted with a compound of formula (I) in vivo. In other circumstances, the cell can be contacted with a compound of formula (I) in vitro.

In another aspect, a method of treating hormone-refractory metastatic prostate cancer in a mammal includes administering to the mammal in need of treatment for hormone-refractory metastatic prostate cancer an effective amount of a compound having the formula (I), or a pharmaceutically acceptable salt thereof. In another aspect, a method of inducing apoptosis in a cell includes contacting the cell with an effective amount of a compound having the formula (I), or a pharmaceutically acceptable salt thereof. In yet another aspect, a method of inducing cell cycle arrest in a cell includes contacting the cell with an effective amount of a compound having the formula (I), or a pharmaceutically acceptable salt thereof. In one aspect, a method of inhibiting the deacetylation of p53 in a cell includes contacting the cell with an effective amount of a compound having the formula (I), or a pharmaceutically acceptable salt thereof. In another aspect, a method of increasing levels of p21 in a cell includes contacting the cell with an effective amount of a compound having the formula (I), or a pharmaceutically acceptable salt thereof. In certain circumstances, the compound of formula (I) can be 7-phenyl-2,4,6-heptatrienoylhydroxamic acid, or a derivative thereof. The method of treating hormone-refractory metastatic prostate cancer in a mammal can include administering to the mammal an effective amount of suberanilo hydoxamic acid, or a pharmaceutically acceptable salt thereof.

The compound formula (I) is: 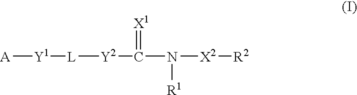
or a pharmaceutically acceptable salt thereof.

In one embodiment, the compound inhibits the deacetylation of p53 in the cell. In another embodiment, the compound increases the levels of p21 in the cell. In yet another embodiment, the compound increases levels of Bax in the cell and may induce cell cycle arrest in the cell. In another embodiment, the compound induces apoptosis in the cell. In certain circumstances, the cell can be contacted with a compound of formula (I) in vivo. In other circumstances, the cell can be contacted with a compound of formula (I) in vitro.

In the compound of formula (I), A can be cyclic moiety selected from the group consisting of C₃₋₁₄ cycloalkyl, 3-14 membered heterocycloalkyl, C₄₋₁₄ cycloalkenyl, 3-14 membered heterocycloalkenyl, monocyclic aryl, or monocyclic heteroaryl; the cyclic moiety being optionally substituted with alkyl, alkenyl, alkynyl, alkoxy, hydroxyl, hydroxylalkyl, halo, haloalkyl, amino, alkylcarbonyloxy, alkyloxycarbonyl, alkylcarbonyl, alkylsulfonylamino, aminosulfonyl, or alkylsulfonyl. For example, A can be C₃₋₈ cycloalkyl, 3-8 membered heterocycloalkyl, C₄₋₈ cycloalkenyl, or 3-8 membered heterocycloalkenyl.

In the compound of formula (I),each of X¹ and X², independently, is O or S and Y¹ can be —CH₂—, —O—, —S—, —N(R^{a})—, —N(R^{a})—C(O)—O—, —O—C(O)—N(R^{a})—, —N(R^{a})—C(O)—N(R^{b})—, —C(O)—O—, —O—C(O)—O—, —N(R^{a})—C(O)—, —C(O)—N(R^{a})—, or a bond. Each of R^{a} and R^{b} independently can be hydrogen, alkyl, alkenyl, alkynyl, alkoxy, hydroxylalkyl, hydroxyl, or haloalkyl. In the compound of formula (I), Y² is a bond.

In the compound of formula (I), L can be an unsaturated straight C₄₋₁₂ hydrocarbon chain containing at least two double bonds, at least one triple bond, or at least one double bond and one triple bond, or a saturated C₄₋₈ hydrocarbon chain; the hydrocarbon chain being optionally substituted with C₁₋₄ alkyl, C₂₋₄ alkenyl, C₂₋₄ alkynyl, C₁₋₄ alkoxy, hydroxyl, halo, carboxyl, amino, nitro, cyano, C₃₋₆ cycloalkyl, 3-6 membered heterocycloalkyl, monocyclic aryl, 5-6 membered heteroaryl, C₁₋₄ alkylcarbonyloxy, C₁₋₄ alkyloxycarbonyl, C₁₋₄ alkylcarbonyl, oxo or formyl. The hydrocarbon chain can be optionally interrupted by —O—, —N(R^{g})—, —N(R^{g})—C(O)—O—, —O—C(O)—N(R^{g})—, —N(R^{g})—C(O)—N(R^{h})—, —O—C(O)—, —C(O)—O—, or —O—C(O)—O—. Each of R^{g} and R^{h}, independently, can be hydrogen, alkyl, alkenyl, alkynyl, alkoxy, hydroxylalkyl, hydroxyl, or haloalkyl;

In the compound of formula (I), R¹ can be hydrogen, alkyl, alkenyl, alkynyl, alkoxy, hydroxylalkyl, hydroxyl, haloalkyl, or an amino protecting group; and R² can be hydrogen, alkyl, hydroxylalkyl, haloalkyl, or a hydroxyl protecting group or a salt thereof.

In certain circumstances, the carbon bonded to Y² is unsaturated, and provided that when L is a C₄₋₅ hydrocarbon chain and contains two double bonds, Y¹ is not CH₂. In certain circumstances, R¹ can be hydrogen, R² can be hydrogen, each of R¹ and R² can be hydrogen, X¹ can be O, X² can be O, each of X¹ and X² can be O, Y¹ can be —CH₂—, —O—, —N(R^{a})—, or a bond, Y¹ can be a bond, L can be unsaturated straight C₄₋₁₀ hydrocarbon chain optionally substituted with C₁₋₄ alkyl, C₂₋₄ alkenyl, C₂₋₄ alkynyl, C₁₋₄ alkoxy, or amino or L can be an unsaturated straight C₅₋₈ hydrocarbon chain optionally substituted with C₁₋₄ alkyl, C₂₋₄ alkenyl, C₂₋₄ alkynyl, C₁₋₄ alkoxy, or amino or L can be an unsubstituted unsaturated straight C₄₋₆ hydrocarbon chain or L can be an unsubstituted unsaturated straight C₅ hydrocarbon chain or L can be an unsubstituted unsaturated straight C₆ hydrocarbon chain or L can be an unsaturated straight C₄₋₁₀ hydrocarbon chain containing 2-5 double bonds optionally substituted with C₁₋₄ alkyl, C₂₋₄ alkenyl, C₂₋₄ alkynyl, or C₁₋₄ alkoxy or L can be an unsaturated straight C₄₋₈ hydrocarbon chain containing 2-5 double bonds optionally substituted with C₁₋₄ alkyl, C₂₋₄ alkenyl, C₂₋₄ alkynyl, or C₁₋₄ alkoxy or L can be —(CH═CH)ₘ— where m is 2 or 3, L being optionally substituted with C₁₋₄ alkyl, C₂₋₄ alkenyl, C₂₋₄ alkynyl, or C₁₋₄ alkoxy or L can be an unsaturated straight C₄₋₁₀ hydrocarbon chain containing 1-2 double bonds and 1-2 triple bonds, the hydrocarbon chain being optionally substituted with C₁₋₄ alkyl, C₂₋₄ alkenyl, C₂₋₄ alkynyl, or C₁₋₄ alkoxy or L can be unsaturated straight C₄₋₈ hydrocarbon chain containing 1-2 double bonds and 1-2 triple bonds, the hydrocarbon chain being optionally substituted with C₁₋₄ alkyl, C₂₋₄ alkenyl, C₂₋₄ alkynyl, or C₁₋₄ alkoxy, or L can be —C≡C—(CH═CH)ₙ— where n is 1 or 2, L being optionally substituted with C₁₋₄ alkyl, C₂₋₄ alkenyl, C₂₋₄ alkynyl, or C₁₋₄ alkoxy.

In certain circumstances, A can be phenyl or A can be phenyl optionally substituted with alkyl, alkenyl, alkynyl, alkoxy, hydroxylalkyl, or amino. In certain circumstances, L can be an unsaturated straight C₄₋₆ hydrocarbon chain or L can be a saturated straight C₆ hydrocarbon chain. In certain circumstances, each of R¹ and R² is hydrogen, each of X¹ and X² is O, or Y¹ can be —CH₂—, —O—, —N(R^{a})—, or a bond.

In certain circumstances, L can be an unsaturated straight C₄₋₈ hydrocarbon chain containing 2-5 double bonds; the hydrocarbon chain being optionally substituted with C₁₋₄ alkyl, C₂₋₄ alkenyl, C₂₋₄ alkynyl, or C₁₋₄ alkoxy or L can be —(CH═CH)ₘ—, where m is 2 or 3, R¹ and R² is hydrogen, each of X¹ and X² is O.

In certain circumstances, Y¹ can be —CH₂—, —O—, —N(R^{a})—, or a bond, L can be an unsaturated straight C₄₋₈ hydrocarbon chain containing 1-2 double bonds and 1-2 triple bonds; the hydrocarbon chain being optionally substituted with C₁₋₄ alkyl, C₂₋₄ alkenyl, C₂₋₄ alkynyl, or C₁₋₄ alkoxy or L can be —C≡C—(CH═CH)ₙ—, where n is 1 or 2, each of R¹ and R² is hydrogen, X¹ and X² is O, Y¹ is —CH₂—, —O—, —N(R^{a})—, or a bond.

Set forth below are examples of compounds of formula (I):5-phenyl-2,4-pentadienoyl hydroxamic acid, N-methyl-5-phenyl-2,4-pentadienoyl hydroxamic acid, 3-methyl-5-phenyl-2,4-pentadienoyl hydroxamic acid, 4-methyl-5-phenyl-2,4-pentadienoyl hydroxamic acid, 4-chloro-5-phenyl-2,4-pentadienoyl hydroxamic acid, 5-(4-dimethylaminophenyl)-2,4-pentadienoyl hydroxamic acid, 5-phenyl-2-en-4-yn-pentanoyl hydroxamic acid, N-methyl-6-phenyl-3,5-hexadienoyl hydroxamic acid, potassium 2-oxo-6-phenyl-3,5-hexadienoate, potassium 2-oxo-8-phenyl-3,5,7-octatrienoate, or 7-phenyl-2,4,6-hepta-trienoylhydroxamic acid. The compound can be 7-phenyl-2,4,6-heptatrienoylhydroxamic acid.

A salt of any of the compounds can be prepared. For example, a pharmaceutically acceptable salt can be formed when an amino-containing compound of formula (I) reacts with an inorganic or organic acid. Some examples of such an acid include hydrochloric acid, hydrobromic acid, hydroiodic acid, sulfuric acid, phosphoric acid, p-bromophenylsulfonic acid, carbonic acid, succinic acid, citric acid, benzoic acid, and acetic acid. Examples of pharmaceutically acceptable salts thus formed include sulfate, pyrosulfate bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, and maleate. A compound of formula (I) may also form a pharmaceutically acceptable salt when a compound having an acid moiety reacts with an inorganic or organic base. Such salts include those derived from inorganic or organic bases, e.g., alkali metal salts such as sodium, potassium, or lithium salts; alkaline earth metal salts such as calcium or magnesium salts; or ammonium salts or salts of organic bases such as morpholine, piperidine, pyridine, dimethylamine, or diethylamine salts.

It should be recognized that a compound can contain chiral carbon atoms. In other words, it may have optical isomers or diastereoisomers.

Alkyl is a straight or branched hydrocarbon chain containing 1 to 10 (preferably, 1 to 6; more preferably 1 to 4) carbon atoms. Examples of alkyl include, but are not limited to, methyl, ethyl, propyl, isopropyl, butyl, isobutyl, sec-butyl, tert-butyl, n-pentyl, 2-methylhexyl, and 3-ethyloctyl.

The terms “alkenyl” and “alkynyl” refer to a straight or branched hydrocarbon chain containing 2 to 10 carbon atoms and one or more (preferably, 1-4 or more preferably 1-2) double or triple bonds, respectively. Some examples of alkenyl and alkynyl are allyl, 2-butenyl, 2-pentenyl, 2-hexenyl, 2-butynyl, 2-pentynyl, and 2-hexynyl.

Cycloalkyl is a monocyclic, bicyclic or tricyclic alkyl group containing 3 to 14 carbon atoms. Some examples of cycloalkyl are cyclopropyl, cyclopentyl, cyclohexyl, cycloheptyl, adamantyl, and norbonyl. Heterocycloalkyl is a cycloalkyl group containing at least one heteroatom (e.g., 1-3) such as nitrogen, oxygen, or sulfur. The nitrogen or sulfur may optionally be oxidized and the nitrogen may optionally be quaternized. Examples of heterocycloalkyl include piperidinyl, piperazinyl, tetrahydropyranyl, tetrahydrofuryl, and morpholinyl. Cycloalkenyl is a cycloalkyl group containing at least one (e.g., 1-3) double bond. Examples of such a group include cyclopentenyl, 1,4-cyclohexa-di-enyl, cycloheptenyl, and cyclooctenyl groups. By the same token, heterocycloalkenyl is a cycloalkenyl group containing at least one heteroatom selected from the group of oxygen, nitrogen or sulfur.

Aryl is an aromatic group containing a 5-14 ring and can contain fused rings, which may be saturated, unsaturated, or aromatic. Examples of an aryl group include phenyl, naphthyl, biphenyl, phenanthryl, and anthracyl. If the aryl is specified as “monocyclic aryl,” if refers to an aromatic group containing only a single ring, i.e., not a fused ring.

Heteroaryl is aryl containing at least one (e.g., 1-3) heteroatom such as nitrogen, oxygen, or sulfur and can contain fused rings. Some examples of heteroaryl are pyridyl, furanyl, pyrrolyl, thienyl, thiazolyl, oxazolyl, imidazolyl, indolyl, benzofuranyl, and benzthiazolyl.

The cyclic moiety can be a fused ring formed from two or more of the just-mentioned groups. Examples of a cyclic moiety having fused rings include fluorenyl, dihydro-dibenzoazepine, dibenzocycloheptenyl, 7H-pyrazino[2,3-c]carbazole, or 9,10-dihydro-9, 10-[2]buteno-anthracene.

Amino protecting groups and hydroxy protecting groups are well-known to those in the art. In general, the species of protecting group is not critical, provided that it is stable to the conditions of any subsequent reaction(s) on other positions of the compound and can be removed without adversely affecting the remainder of the molecule. In addition, a protecting group may be substituted for another after substantive synthetic transformations are complete. Examples of an amino protecting group include, but not limited to, carbamates such as 2,2,2-trichloroethylcarbamate or tertbutylcarbamate. Examples of a hydroxyl protecting group include, but not limited to, ethers such as methyl, t-butyl, benzyl, p-methoxybenzyl, p-nitrobenzyl, allyl, trityl, methoxymethyl, 2-methoxypropyl, methoxyethoxymethyl, ethoxyethyl, tetrahydropyranyl, tetrahydrothiopyranyl, and trialkylsilyl ethers such as trimethylsilyl ether, triethylsilyl ether, dimethylarylsilyl ether, triisopropylsilyl ether and t-butyldimethylsilyl ether; esters such as benzoyl, acetyl, phenylacetyl, formyl, mono-, di-, and trihaloacetyl such as chloroacetyl, dichloroacetyl, trichloroacetyl, trifluoroacetyl; and carbonates including but not limited to alkyl carbonates having from one to six carbon atoms such as methyl, ethyl, n-propyl, isopropyl, n-butyl, t-butyl; isobutyl, and n-pentyl; alkyl carbonates having from one to six carbon atoms and substituted with one or more halogen atoms such as 2,2,2-trichloroethoxymethyl and 2,2,2-trichloro-ethyl; alkenyl carbonates having from two to six carbon atoms such as vinyl and allyl; cycloalkyl carbonates having from three to six carbon atoms such as cyclopropyl, cyclobutyl, cyclopentyl and cyclohexyl; and phenyl or benzyl carbonates optionally substituted on the ring with one or more C₁₋₆ alkoxy, or nitro. Other protecting groups and reaction conditions can be found in T. W. Greene, Protective Groups in Organic Synthesis, (3rd, 1999, John Wiley & Sons, New York, N.Y.).

Note that an amino group can be unsubstituted (i.e., —NH₂), mono-substituted (i.e., —NHR), or di-substituted (i.e., —NR₂). It can be substituted with groups (R) such as alkyl, cycloalkyl, heterocycloalkyl, aryl, heteroaryl, aralkyl, or heteroaralkyl. Halo refers to fluoro, chloro, bromo, or iodo.

Other features or advantages will be apparent from the following detailed description of several embodiments, and also from the appended claims.

### DETAILED DESCRIPTION

HDAC inhibitors with potent and specific HDAC inhibitory activity can be used to target specific HDACs, which in turn, can affect acetylation of proteins other than histones. For example, in addition to histones, HDACs can deacetylate other proteins such as the tumor suppressor, p53. Human p53 functions as a central integrator of signals arising from different forms of cellular stress, including DNA damage, hypoxia, nucleotide deprivation, and oncogene activation (Prives, Cell (1998) 95:5-8). In response to these signals, p53 protein levels are greatly increased with the result that the accumulated p53 activates pathways of cell cycle arrest or apoptosis depending on the nature and strength of these signals. One clearly important aspect of p53 function is its activity as a gene-specific transcriptional activator. Among the genes with known p53-response elements are several with well-characterized roles in either regulation of the cell cycle or apoptosis, including GADD45, p21/Waf1/Cip1, cyclin G, Bax, IGF-BP3, and MDM2 (Levine, Cell (1997) 88:323-331).

The inhibition of HDAC activity thus represents a novel approach for intervening in cell cycle regulation and that HDAC inhibitors have great therapeutic potential in the treatment of cell proliferative diseases or conditions. To date, only a few inhibitors of histone deacetylase are known in the art. Richon et al., Proc. Natl. Acad. Sci. USA, 95: 3003-3007 (1998), discloses that HDAC activity is inhibited by trichostatin A (TSA), a natural product isolated from Streptomyces hygroscopicus, and by a synthetic compound, suberoylanilide hydroxamic acid (SAHA). Yoshida and Beppu, Exper. Cell Res., 177: 122-131 (1988), teaches that TSA causes arrest of rat fibroblasts at the G1 and G2 phases of the cell cycle, implicating HDAC in cell cycle regulation. Finnin et al., Nature, 401: 188-193 (1999), teaches that TSA and SAHA inhibit cell growth, induce terminal differentiation, and prevent the formation of tumors in mice. While the effects of TSA are potent, the production of TSA is costly and highly inefficient (Ruijter et al., Biochem J., 370:737-49 (2003)). It has further been reported that class I and class II HDACs are inhibited differently by HDAC inhibitors (Ruijter et al., Biochem J., 370:737-49 (2003)).

A pharmaceutical composition can be used to inhibit histone deacetylase in cells. In one embodiment, the composition can be used in a method for inhibiting histone deacetylase activities of HDAC1 or HDAC2. The compounds of formula (I) can stabilize the acetylation of p53. In one embodiment, the acetylation of p53 is unexpectedly stabilized at Lysine residues 373 and 382 but not at Lysine 320. In a further embodiment, the increased or stabilized acetylation of p53 may lead to a p53 dependent increase in p21 levels and/or may lead to activation of Bax which surprisingly results in cell cycle arrest or apoptosis. Unexpectedly, compounds of formula (I) inhibit HDAC1, resulting in p53 independent activation of p21.

A pharmaceutical composition including a compound of formula (I) can be used preferably to treat hormone refractory metastatic disease. Current therapies for prostate cancer include hormone manipulation such as orchidectomy and/or medical castration using anti-androgen and LHRH analogues or oestrogens. Both early and late stages of prostate cancer can be treated with anti-androgens such as flutamide or casodex. While initially successful, anti-androgen therapy often fails, leading to hormone refractory metastatic disease. Pharmaceutical compounds of formula (I) can be used together with anti-androgen therapy or used alone in early or late stages of prostate cancer. Pharmaceutical compounds of formula (I) can be used concurrently with chemotherapy treatments such as cyclophosphamide, estramustine, doxorubicin, mitoxantrone, cisplatin, etoposide or taxol. Examples of pharmaceutical compositions that can be used to treat prostate cancer can include 7-phenyl-2,4,6-heptatrienoylhydroxamic acid or suberanilo hydoxamic acid (SAHA) (see for example, Richon et al., Proc. Natl. Acad. Sci. USA, 95: 3003-3007 (1998), herein incorporated by reference in its entirety).

A carboxylic acid-containing compound of formula (I) can be prepared by any known methods in the art. For example, a compound of formula (I) having an unsaturated hydrocarbon chain between A and —C(═X¹)— can be prepared according to the following scheme:

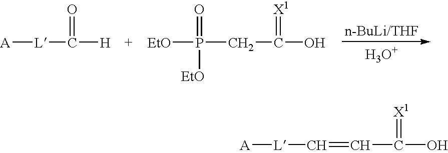

where L′ is a saturated or unsaturated hydrocarbon linker between A and —CH═CH— in a compound of formula (I), and A and X¹ has the same meaning as defined above. See Coutrot et al., Syn. Comm. 133-134 (1978). Briefly, butyllithium was added to an appropriate amount of anhydrous tetrahydrofuran (THF) at a very low temperature (e.g., −65° C.). A second solution having diethylphosphonoacetic acid in anhydrous THF was added dropwise to the stirred butyllithium solution at the same low temperature. The resulting solution is stirred at the same temperature for an additional 30-45 minutes which is followed by the addition of a solution containing an aromatic acrylaldehyde in anhydrous THF over 1-2 hours. The reaction mixture is then warmed to room temperature and stirred overnight. It is then acidified (e.g., with HCl) which allows the organic phase to be separated. The organic phase is then dried, concentrated, and purified (e.g., by recrystallization) to form an unsaturated carboxylic acid-containing intermediate.

Alternatively, a carboxylic acid-containing compound can be prepared by reacting an acid ester of the formula A-L′-C(═O)—O-lower alkyl with a Grignard reagent (e.g., methyl magnesium iodide) and a phosphorus oxychloride to form a corresponding aldehyde, which can be further oxidized (e.g., by reacting with silver nitrate and aqueous NaOH) to form an unsaturated carboxylic acid-containing intermediate.

Other types of carboxylic acid-containing compounds (e.g., those containing a linker with multiple double bonds or triple bonds) can be prepared according to published procedures such as those described in Parameswara et al., Synthesis, 815-818 (1980) and Denny et al., J. Org. Chem., 27, 3404 (1962).

Carboxylic acid-containing compounds described above can then be converted to hydroxamic acid-containing compounds according to the following scheme:

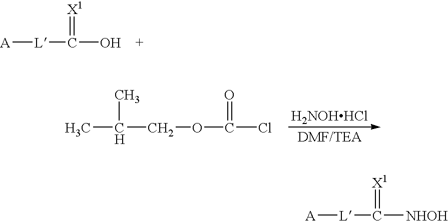

Triethylamine (TEA) is added to a cooled (e.g., 0-5° C.) anhydrous THF solution containing the carboxylic acid. Isobutyl chloroformate is then added to the solution having carboxylic acid, which is followed by the addition of hydroxylamine hydrochloride and TEA. After acidification, the solution was filtered to collect the desired hydroxamic acid-containing compounds.

An N-substituted hydroxamic acid can be prepared in a similar manner as described above. A corresponding carboxylic acid A-L′-C(═O)—OH can be converted to an acid chloride by reacting with oxalyl chloride (in appropriate solvents such as methylene chloride and dimethylformamide), which in turn, can be converted to a desired N-substituted hydroxamic acid by reacting the acid chloride with an N-substituted hydroxylamine hydrochloride (e.g., CH₃NHOH.HCl) in an alkaline medium (e.g., 40% NaOH (aq)) at a low temperature (e.g., 0-5° C.). The desired N-substituted hydroxamic acid can be collected after acidifying the reaction mixture after the reaction has completed (e.g., in 2-3 hours).

As to compounds of formula (I) in which X¹ is S, the compounds can be prepared according to procedures described in Sandler, S. R. and Karo, W., Organic Functional Group Preparations, Volume III (Academic Press, 1972) at pages 436-437. For preparation of compounds of formula (I) wherein X² is —N(R^{c})OH— and X¹ is S, see procedures described in U.S. Pat. Nos. 5,112,846; 5,075,330 and 4,981,865.

Compounds of formula (I) containing an α-keto acid moiety (e.g., when X¹ is oxygen and X² is —C(═O)OM or A-L′-C(═O)—C(═O)—OM, where A and L′ have been defined above and M can be hydrogen, lower alkyl or a cation such as K⁺), these compounds can be prepared by procedures based on that described in Schummer et al., Tetrahedron, 43, 9019 (1991). Briefly, the procedure starts with a corresponding aldehyde-containing compound (e.g., A-L′-C(═O)—H), which is allowed to react with a pyruvic acid in a basic condition (KOH/methanol) at a low temperature (e.g., 0-5° C.). Desired products (in the form of a potassium salt) are formed upon warming of the reaction mixture to room temperature.

The compounds described above, as well as their (thio)hydroxamic acid or α-keto acid counterparts, can possess histone deacetylase inhibitory properties.

Note that appropriate protecting groups may be needed to avoid forming side products during the preparation of a compound of formula (I). For example, if the linker L′ contains an amino substituent, it can be first protected by a suitable amino protecting group such as trifluoroacetyl or tert-butoxycarbonyl prior to being treated with reagents such as butyllithium. See, e.g., T. W. Greene, supra, for other suitable protecting groups.

A compound produced by the methods shown above can be purified by flash column chromatography, preparative high performance liquid chromatography, or crystallization.

An effective amount is defined as the amount which is required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardley, N.Y., 537 (1970). An effective amount of a compound described herein can range from about 1 mg/kg to about 300 mg/kg. Effective doses will also vary, as recognized by those skilled in the art, dependant on route of administration, excipient usage, and the possibility of co-usage, pre-treatment, or post-treatment, with other therapeutic treatments including use of other chemotherapeutic agents and radiation therapy. Other chemotherapeutic agents that can be co-administered (either simultaneously or sequentially) include, but not limited to, paclitaxel and its derivatives (e.g., taxotere), doxorubicin, L-asparaginase, dacarbazine, amascrine, procarbazine, hexamethylmelamine, mitoxantrone, and gemicitabine.

The pharmaceutical composition may be administered via the parenteral route, including orally, topically, subcutaneously, intraperitoneally, intramuscularly, and intravenously. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. Because some of the compounds described herein can have limited water solubility, a solubilizing agent can be included in the composition to improve the solubility of the compound. For example, the compounds can be solubilized in polyethoxylated castor oil (Cremophor EL®) and may further contain other solvents, e.g., ethanol. Furthermore, compounds described herein can also be entrapped in liposomes that may contain tumor-directing agents (e.g., monoclonal antibodies having affinity towards tumor cells).

A compound described herein can be formulated into dosage forms for other routes of administration utilizing conventional methods. For example, it can be formulated in a capsule, a gel seal, or a tablet for oral administration. Capsules may contain any standard pharmaceutically acceptable materials such as gelatin or cellulose. Tablets may be formulated in accordance with conventional procedures by compressing mixtures of a compound described herein with a solid carrier and a lubricant. Examples of solid carriers include starch and sugar bentonite. Compounds of this invention can also be administered in a form of a hard shell tablet or a capsule containing a binder, e.g., lactose or mannitol, a conventional filler, and a tableting agent.

The activities of a compound described herein can be evaluated by methods known in the art, e.g., MTT (3-[4,5-dimehtythiazol-2-yl]-2,5-diphenyltetrazolium bromide) assay, clonogenic assay, ATP assay, or Extreme Drug Resistance (EDR) assay. See Freuhauf, J. P. and Manetta, A., Chemosensitivity Testing in Gynecologic Malignancies and Breast Cancer 19, 39-52 (1994). The EDR assay, in particular, is useful for evaluating the antitumor and antiproliferative activity of a compound of this invention. Cells are treated for four days with compound of formula (I). Both untreated and treated cells are pulsed with tritiated thymidine for 24 hours. Radioactivity of each type of cells is then measured and compared. The results are then plotted to generate drug response curves, which allow IC₅₀ values (the concentration of a compound required to inhibit 50% of the population of the treated cells) to be determined.

The histone acetylation activity of a compound described herein can be evaluated in an assay using mouse erythroleukemia cells. Studies are performed with the DS19 mouse erythroleukemia cells maintained in RPMI 1640 medium with 25 mM HEPES buffer and 5% fetal calf serum. The cells are incubated at 37° C.

Histones are isolated from cells after incubation for periods of 2 and 24 hours. The cells are centrifuged for 5 minutes at 2000 rpm in the Sorvall SS34 rotor and washed once with phosphate buffered saline. The pellets are suspended in 10 ml lysis buffer (10 mM Tris, 50 mM sodium bisulfite, 1% Triton X-100, 10 mM magnesium chloride, 8.6% sucrose, pH 6.5) and homogenized with six strokes of a Teflon pestle. The solution is centrifuged and the pellet washed once with 5 ml of the lysis buffer and once with 5 ml 10 mM Tris, 13 mM EDTA, pH 7.4. The pellets are extracted with 2×1 mL 0.25N HCl. Histones are precipitated from the combined extracts by the addition of 20 mL acetone and refrigeration overnight. The histones are pelleted by centrifuging at 5000 rpm for 20 minutes in the Sorvall SS34 rotor. The pellets are washed once with 5 mL acetone and protein concentration are quantitated by the Bradford procedure.

Separation of acetylated histones is usually performed with an acetic acid-urea polyacrylamide gel electrophoresis procedure. Resolution of acetylated H4 histones is achieved with 6,25N urea and no detergent as originally described by Panyim and Chalkley, Arch. Biochem. Biophys. 130, 337-346 (1969). 25 μg total histones are applied to a slab gel which is run at 20 ma. The run is continued for a further two hours after the Pyronon Y tracking dye has run off the gel. The gel is stained with Coomassie Blue R. The most rapidly migrating protein band is the unacetylated H4 histone followed by bands with 1, 2, 3 and 4 acetyl groups which can be quantitated by densitometry. The procedure for densitometry involves digital recording using the Alpha Imager 2000, enlargement of the image using the PHOTOSHOP program (Adobe Corp.) on a MACINTOSH computer (Apple Corp.), creation of a hard copy using a laser printer and densitometry by reflectance using the Shimadzu CS9000U densitometer. The percentage of H4 histone in the various acetylated states is expressed as a percentage of the total H4 histone.

The concentration of a compound of formula (I) required to decrease the unacetylated H4 histone by 50% (i.e., EC₅₀) can then be determined from data obtained using different concentrations of test compounds.

Histone deacetylase inhibitory activity can be measured based on procedures described by Hoffmann et al., Nucleic Acids Res., 27, 2057-2058 (1999). Briefly, the assay starts with incubating the isolated histone deacetylase enzyme with a compound of formula (I), followed by the addition of a fluorescent-labeled lysine substrate (contains an amino group at the side chain which is available for acetylation). HPLC is used to monitor the labeled substrate. The range of activity of each test compound is preliminarily determined using results obtained from HPLC analyses. IC₅₀ values can then be determined from HPLC results using different concentrations of compounds of this invention. All assays are duplicated or triplicated for accuracy. The histone deacetylase inhibitory activity can be compared with the increased activity of acetylated histone for confirmation.

The toxicity of a compound described herein is evaluated when a compound of formula (I) is administered by single intraperitoneal dose to test mice. After administration of a predetermined dose to three groups of test mice and untreated controls, mortality/morbidity checks are made daily. Body weight and gross necropsy findings are also monitored. For reference, see Gad, S. C. (ed.), Safety Assessment for Pharmaceuticals (Van Nostrand Reinhold, New York, 1995).

Without further elaboration, it is believed that one skilled in the art can, based on the description herein, utilize the present invention to its fullest extent. The following specific examples, which described syntheses, screening, and biological testing of various compounds of formula (I), are therefore, to be construed as merely illustrative, and not limitative of the remainder of the disclosure in any way whatsoever. All publications recited herein, including patents, are hereby incorporated by reference in their entirety.

### EXAMPLE 1

### Synthesis of 7-phenyl-2,4,6-heptatrienoylhydroxamic acid

Triethylamine (TEA, 24.1 mL) was added to a cooled (0-5° C.) solution of 7-phenyl-2,4,6-heptatrienoic acid (27.8 g) in 280 mL of anhydrous dimethylformamide. To this solution was added dropwise isobutyl chloroformate (22.5 mL) over a period of 75 minutes. The reaction mixture was stirred for 40 minutes and hydroxylamine hydrochloride (24.2 g) was added followed by dropwise addition of 48 mL of TEA over a period of 70 minutes at 0-5° C. The reaction was allowed to warm to room temperature and stirred overnight. To the stirred reaction mixture at room temperature was added 280 mL of a 1% (by weight) solution of citric acid followed by 1050 mL of water. The mixture was stirred for 30 minutes and then filtered. The filtered cake was washed with water (200 mL) and dried under vacuum to afford 20.5 g of the desired 7-phenyl-2,4,6-heptatrienoylhydroxamic acid. ¹H NMR (DMSO-d₆, 300 MHz), δ(ppm) 7.48 (m, 2H), 7.32 (m, 2H), 7.19 (m, 2H), 7.01 (m, 1H), 6.75 (m, 2H), 6.51 (m, 1H), 5.93 (d, 1H).

### EXAMPLE 2

### Synthesis of 3-methyl-5-phenyl-2,4-pentadienoic acid

To a cooled (−10 to −5° C.) 165 mL of 3 M solution of methyl magnesium iodide in ether was added dropwise a solution of ethyl trans-cinnamate (25.0 g) in 200 mL of anhydrous ether. The reaction was warmed to room temperature and stirred overnight. The mixture was then heated up to 33° C. under reflux for two hours and cooled to 0° C. A white solid was formed during cooling and water (105 mL) was gradually added to dissolve the white precipitate followed by an additional 245 mL of saturated aqueous ammonium chloride solution. The mixture was then stirred until the solids were completely dissolved and extracted with 100 mL of ether three times. The combined extract was washed with 100 mL of water, dried over anhydrous sodium sulfate and filtered. The solvent was evaporated to give 22.1 g of the desired 4-phenyl-2-methyl-3-buten-2-ol as an oil which was used in the next step without further purification. ¹H NMR (CDCl₃, 300 MHz), δ(ppm) 7.41 (m, 5H), 6.58 (d, 1H), 6.34 (d, 1H), 1.41 (broad s, 6H).

Dimethylformamide (DMF, anhydrous, 25 mL) was cooled to 0-5° C. and phosphorus oxychloride (16.4 mL) was added dropwise over a period of an hour. The resulting solution was added dropwise to a cooled (0-5° C.) solution of 4-phenyl-2-methyl-3-buten-2-ol (0.14 mol) in 60 mL of anhydrous DMF over a period of an hour. The reaction mixture was then warmed to room temperature, gradually heated up to 80° C., stirred at 80° C. for three hours and cooled to 0-5° C. To the cooled reaction solution was added dropwise a solution of sodium acetate (80 g) in deionized water (190 mL) over a period of two hours. The mixture was then reheated to 80° C., stirred at 80° C. for an additional 10 minutes, cooled down to room temperature and extracted with ether (300 mL) twice. The combined extract was washed with water (200 mL), dried over anhydrous sodium sulfate, filtered and concentrated in vacuum to yield 16.7 g of the desired 3-methyl-5-phenyl-2,4-pentadienal as a liquid which was used in the next step without further purification.

To a stirred solution of 3-methyl-5-phenyl-2,4-pentadienal (16.5 g) in ethanol (330 mL) was added dropwise a solution of silver nitrate (19.28 g) in water (160 mL) followed by dropwise addition of an aqueous sodium hydroxide (25 g, 80 mL) solution. The resulting mixture was allowed to stir for an additional five hours and then filtered. The solid was washed with ethanol. The combined filtrate was concentrated in vacuum. The residue was dissolved in water (200 mL). The aqueous solution was extracted with ether (300 mL) twice and acidified with 6 N hydrochloric acid (74 mL). The solid formed was filtered and recrystallized from methanol (40 mL) to yield 2.65 g of the desired 3-methyl-5-phenyl-2,4-pentadienoic acid. ¹ H NMR (acetone-d₆, 300 MHz), δ(ppm) 7.60 (d, 2H), 7.35 (m, 3H), 7.06 (m, 2H), 6.02 (broad s, 1H), 2.50 (s, 3H).

### EXAMPLE 3

### Synthesis of 4-methyl-5-phenyl-2,4-pentadienoic acid

Butyllithium (135 mL of 2.5 N solution) was added to 600 mL of anhydrous tetrahydrofuran (THF) at −65° C. A solution of diethylphosphonoacetic acid (30.5 g) in 220 mL of anhydrous THF was added dropwise to the stirred solution at −65° C. over a period of 60 minutes. The resulting solution was stirred at −65° C. for an additional 30 minutes and then a solution of α-methyl-trans-cinnamaldehyde (23.2 g) in 100 mL of anhydrous THF was added to the reaction at −65° C. over a period of 70 minutes. The reaction was stirred for one hour, allowed to warm to room temperature and then stirred overnight. The reaction was then acidified with 5% hydrochloric acid (125 mL) to a pH of 2.8. The aqueous layer was extracted with 100 mL of ether twice and with 100 mL of ethyl acetate once. The combined organic extract was dried over anhydrous sodium sulfate, filtered and concentrated under vacuum. The crude material was dissolved in 100 mL of hot methanol and then refrigerated overnight. The crystals formed were filtered and dried under vacuum to afford 25.8 g of the desired 4-methyl-5-phenyl-2,4-pentadienoic acid. ¹H NMR (acetone-d₆, 300 MHz), δ(ppm) 7.53 (d, 1H), 7.43 (m, 4H), 7.37 (dd, 1H), 6.97 (broad s, 1H), 6.02 (d, 1H), 2.07 (s, 3H).

### EXAMPLE 4

### Synthesis of 4-chloro-5-phenyl-2,4-pentadienoic acid

Butyllithium (50 mL of 2.5 N solution) was added to 250 mL of anhydrous tetrahydrofuran (THF) at −65° C. A solution of diethylphosphonoacetic acid (11.4 g) in 90 mL of anhydrous THF was added dropwise to the stirred solution at −65° C. The resulting solution was stirred at −65° C. for an additional 40 minutes and then a solution of α-chloro-cinnamaldehyde (10.0 g) in 60 mL of anhydrous THF was added to the reaction at −65° C. over a period of 95 minutes. The reaction was stirred for one hour, allowed to warm to room temperature and then stirred overnight. The reaction was then acidified with 5% hydrochloric acid (48 mL) to a pH of 3.9. The aqueous layer was extracted with 50 mL of ether twice and with 50 mL of ethyl acetate once. The combined organic extract was dried over anhydrous sodium sulfate, filtered and concentrated under vacuum. The crude material was dissolved in 30 mL of hot methanol and then refrigerated overnight. The crystals formed were filtered and dried under vacuum to afford 9.2 g of the desired 4-chloro-5-phenyl-2,4-pentadienoic acid. ¹H NMR (acetone-d₆, 300 MHz), δ(ppm) 7.86 (d, 2H), 7.60 (d, 1H), 7.45 (m, 3H), 7.36 (broad s, 1H), 6.32 (d, 1H).

### EXAMPLE 5

### Synthesis of 5-phenyl-2-ene-4-pentynoic acid

Butyllithium (16 mL of 2.5 N solution) was added to 75 mL of anhydrous tetrahydrofuran (THF) at −65° C. A solution of diethylphosphonoacetic acid (3.6 g) in 25 mL of anhydrous THF was added dropwise to the stirred solution at −65° C. over a period of 15 minutes. The resulting solution was stirred at −65° C. for an additional 30 minutes and then a solution of phenylpropargyl aldehyde (2.5 g) in 20 mL of anhydrous THF was added to the reaction at −65° C. over a period of 20 minutes. The reaction was stirred for one hour, allowed to warm to room temperature and then stirred overnight. The reaction was then acidified with 6 N hydrochloric acid (5 mL) to a pH of 1.0. The aqueous layer was extracted with 75 mL of ethyl acetate three times. The combined organic extract was dried over anhydrous sodium sulfate, filtered and concentrated under vacuum. The crude material was recrystallized with chloroform:ether (90:10) and then refrigerated overnight. The crystals were filtered and dried under vacuum to afford 1.1 g of the desired 5-phenyl-2-ene-4-pentynoic acid. ¹H NMR (acetone-d₆, 300 MHz), δ(ppm) 7.50 (m, 5H), 6.98 (d, 1H), 6.35 (d, 1H).

### EXAMPLE 6

### Synthesis of 5-(p-dimethylaminophenyl)-2,4-pentadienoic acid

Butyllithium (24 mL of 2.5 N solution) was added to 120 mL of anhydrous tetrahydrofuran (THF) at −65° C. A solution of diethylphosphonoacetic acid (5.5 g) in 45 mL of anhydrous THF was added dropwise to the stirred solution at −65° C. over a period of one hour. The resulting solution was stirred at −65° C. for an additional 30 minutes and then a solution of p-dimethylaminocinnamaldehyde (5.0 g) in 80 mL of anhydrous THF was added to the reaction at −65° C. over a period of 30 minutes. The reaction was stirred for one hour, allowed to warm to room temperature and then stirred overnight. The reaction was then quenched with 400 mL of water and extracted with 300 mL of ethyl acetate three times. The aqueous layer was acidified with 5% hydrochloric acid (11 mL) to a pH of 6.1. The solid formed was filtered, washed with 75 mL of water and dried to yield 3.83 g of the desired 5-(p-dimethylaminophenyl)-2,4-pentadienoic acid. ¹H NMR (DMSO-d₆, 300 MHz), δ(ppm) 7.34 (m, 3H), 6.82 (m, 2H), 6.70 (d, 2H), 5.84 (d, 1H), 2.94 (s, 6H).

### EXAMPLE 7

### Synthesis of 5-(2-furyl)-2,4-pentadienoic acid

Butyllithium (70 mL of 2.5 N solution) was added to 350 mL of anhydrous tetrahydrofuran (THF) at −65° C. A solution of diethylphosphonoacetic acid (15.9 g) in 130 mL of anhydrous THF was added dropwise to the stirred solution at −65° C. over a period of 75 minutes. The resulting solution was stirred at −65° C. for an additional 30 minutes and then a solution of trans-3-(2-furyl)acrolein (10.0 g) in 85 mL of anhydrous THF was added to the reaction at −65° C. over a period of 2 hours. The reaction was allowed to warm to room temperature and stirred overnight. The reaction was then acidified with 5% hydrochloric acid (85 mL) to a pH of 3.5 followed by addition of 30 mL of water. The aqueous layer was extracted with 50 mL of ether twice and with 50 mL of ethyl acetate once. The combined organic extract was dried over anhydrous sodium sulfate, filtered and concentrated under vacuum to give an oil. The crude oil was dissolved in 45 mL of hot methanol and then refrigerated overnight. The crystals formed were filtered and dried under vacuum to afford 9.2 g of the desired 5-(2-furyl)-2,4-pentadienoic acid. ¹H NMR (acetone-d₆, 300 MHz), δ(ppm) 7.64 (broad s, 1H), 7.42 (m, 1H), 6.86 (m, 2H), 6.58 (m, 2H), 6.05 (d, 1H).

### EXAMPLE 8

### Synthesis of 6-phenyl-3,5-hexadienoic acid

Triphenylphosphine (178.7 g) and 3-chloropropionic acid (73.9 g) were mixed in a 1-liter 3-neck round bottom flask equipped with a mechanical stirrer, reflux condenser with a nitrogen inlet and a thermocouple. The mixture was heated to 145° C. under nitrogen and stirred for 2 hours. The reaction was then cooled to 70° C. Ethanol (550 mL) was added and the mixture was refluxed at 80° C. until complete dissolution. The solution was cooled to room temperature and ether (900 mL) was added. The mixture was placed in the freezer overnight. The solids were collected by filtration and dried under vacuum to afford 217 g of 3-(triphenylphosphonium)propionic acid chloride as a white solid which was used in the next step without further purification.

Sodium hydride (12.97 g) in an oven dried 5-liter 3-neck round bottom flask equipped with a mechanical stirrer and a thermocouple was cooled to 0-5° C. in an ice bath. A solution of 3-(triphenylphosphonium)propionic acid chloride (100.0 g) and trans-cinnamaldehyde (34 mL) in 400 mL each of anhydrous dimethyl sulfoxide and tetrahydrofuran was added over a period of 3 hours. The reaction was then allowed to warm to room temperature and stirred overnight. The reaction mixture was cooled to 0-5° C. in an ice bath and water (1.6 liters) was added dropwise. The aqueous solution was acidified with 12 N hydrochloric acid (135 mL) to a pH of 1 and extracted with ethyl acetate (1.6 liters) twice. The combined organic layers was washed with water (1000 mL) three times, dried over anhydrous sodium sulfate and concentrated under vacuum to afford a yellow oil. The crude oil was dissolved in 125 mL of methylene chloride and chromatographed on a Biotage 75L silica gel column and eluted with methylene chloride:ether (9:1). The fractions containing the desired product were combined and the solvents were removed under vacuum to afford 10.38 g of 6-phenyl-3,5-hexadienoic acid. ¹H NMR (CDCl₃, 300 MHz), δ(ppm) 7.33 (m, 5H), 6.80 (m, 1H), 6.53 (d, 1H), 6.34 (m, 1H), 5.89 (m, 1H), 3.25 (d, 2H).

### EXAMPLE 9

### Synthesis of 8-phenyl-3,5,7-octatrienoic acid

A solution of 5-phenyl-2,4-pentadienal (15 g) and 3-(triphenylphosphonium)-propionic acid chloride (35.2 g) in 140 mL each of anhydrous tetrahydrofuran and anhydrous dimethyl sulfoxide was added dropwise to sodium hydride (4.6 g) at 0-5° C. under nitrogen over a period of four hours. The reaction was allowed to warm to room temperature and stirred overnight. The reaction mixture was cooled to 0-5° C. and water (280 mL) was added dropwise over a period of 30 minutes. The aqueous layer was extracted with ethyl acetate (280 mL) twice, acidified with 12 N hydrochloric acid (24 mL) to a pH of 1, extracted again with ethyl acetate (280 mL) twice. The combined organic layers were washed with water (500 mL) twice, dried over anhydrous sodium sulfate and concentrated under vacuum to give an oil. The oily crude product was chromatographed on a Biotage 40M silica gel column and eluted with methylene chloride:ethyl acetate (95:5). The fractions containing the desired product were combined and the solvents were removed under vacuum to afford 0.7 g of 8-phenyl-3,5,7-octatrienoic acid. ¹H NMR (acetone-d₆, 300 MHz), δ(ppm) 7.46 (m, 2H), 7.26 (m, 3H), 6.95 (m, 1H), 6.60 (d, 1H), 6.34 (m, 3H), 5.87 (m, 1H), 3.17 (d, 2H).

### EXAMPLE 10

### Synthesis of potassium 2-oxo-6-phenyl-3,5-hexadienoate

A solution of trans-cinnamaldehyde (26.43 g) and pyruvic acid (11.9 mL) in 10 mL of methanol was stirred and chilled to 0-5° C. in an ice bath. To the chilled solution was added 35 mL of potassium hydroxide (16.83 g in 50 mL of methanol) over a period of 20 minutes. The remaining methanolic potassium hydroxide was added rapidly and the ice bath was removed. The solution changed from a yellow to a dark orange and the precipitate was formed. The reaction mixture was chilled in the refrigerator overnight and the solid was collected by filtration, washed with 50 mL of methanol three times, 50 mL of ether and then air dried to afford 29.3 g of the desired 2-oxo-6-phenyl-3,5-hexadienoate as a yellow solid (61.0%). ¹H NMR (DMSO-d₆/D₂O, 300 MHz), δ(ppm) 7.48 (d, 2H), 7.28 (m, 4H), 7.12 (d, 2H), 6.27 (d, 1H).

### EXAMPLE 11

### Synthesis of potassium 2-oxo-8-phenyl-3,5,7-octatrienoate

To a cooled (0-55° C.) 927 mL of 1 M solution of phenyl magnesium bromide in tetrahydofuran was added dropwise a solution of crotonaldehyde (65.0 g) in 130 mL of anhydrous ether over a period of 2 hours and 45 minutes. The reaction was stirred for an additional 45 minutes and then warmed to room temperature. After four more hours of stirring, saturated ammonium chloride aqueous solution (750 mL) was added to the reaction. The mixture was extracted with 750 mL of ether twice. The combined extract was dried over anhydrous potassium carbonate and filtered. The solvent was evaporated to give 135.88 g (99.9%) of the desired 1-phenyl-2-buten-1-ol as an oil which was used in the next step without further purification.

1-Phenyl-2-buten-1-ol (135.88 g) was dissolved in 2300 mL of dioxane and treated with 2750 mL of dilute hydrochloric acid (2.3 mL of concentrated hydrochloric acid in 2750 mL of water) at room temperature. The mixture was stirred overnight and then poured into 4333 mL of ether and neutralized with 2265 mL of saturated sodium bicarbonate. The aqueous phase was extracted with 1970 mL of ether. The combined extract was dried over anhydrous potassium carbonate. Evaporation of the solvent followed by Kugelrohr distillation at 30° C. for 30 minutes afforded 131.73 g (96.8%) of the desired 4-phenyl-3-buten-2-ol as an oil which was used in the next step without further purification.

Dimethylformamide (DMF, anhydrous, 14 mL) was cooled to 0-5° C. and phosphorus oxychloride (8.2 mL) was added dropwise over a period of 40 minutes. The resulting solution was added dropwise to a cooled (0-5° C.) solution of 4-phenyl-3-buten-2-ol (10 g) in 32 mL of anhydrous DMF over a period of an hour. The reaction mixture was warmed to room temperature over a 35-minute period and then gradually heated up to 80° C. over a period of 45 minutes. The reaction was stirred at 80° C. for three hours and then cooled to 0-5° C. To the cooled reaction solution was added dropwise a solution of sodium acetate (40 g) in deionized water (100 mL) over a period of one hour. The mixture was then reheated to 80° C., stirred at 80° C. for an additional 10 minutes, cooled down to room temperature and extracted with ether (100 mL) twice. The combined extract was washed with brine (100 mL), dried over anhydrous sodium sulfate, filtered and concentrated under vacuum to yield 8.78 g of the desired 5-phenyl-2,4-pentadienal as a liquid which was used in the next step without further purification. ¹H NMR (CDCl₃, 300 MHz), δ(ppm) 7.51 (m, 2H), 7.37 (m, 3H), 7.26 (m, 1H), 7.01 (m, 2H), 6.26 (m, 1H).

A solution of 5-phenyl-2,4-pentadienal (6.70 g) and pyruvic acid (3.0 mL) in 5 mL of methanol was stirred and chilled to 0-5° C. in an ice bath. To the chilled solution was added a solution of 35 mL of potassium hydroxide (3.5 g) in 10 mL of methanol dropwise over a period of 30 minutes. The remaining methanolic potassium hydroxide was added rapidly and the ice bath was removed. The reaction was allowed to warm to room temperature and stirred for another hour. The flask was then refrigerated overnight. The solid was collected by filtration, washed with 15 mL of methanol three times, 15 mL of ether and then air dried to afford 6.69 g of potassium 2-oxo-8-phenyl-3,5,7-octatrienoate as a yellow solid. ¹H NMR (DMSO-d₆, 300 MHz), δ(ppm) 7.52 (d, 2H), 7.32 (m, 3H), 7.10 (m, 2H), 6.83 (dd, 2H), 6.57 (dd, 1H), 6.13 (d, 1H).

### EXAMPLE 12

### Synthesis of cinnamoylhydroxamic acid

Triethylamine (TEA, 17.6 mL) was added to a cooled (0-5° C.) solution of trans-cinnamic acid (15.0 g) in 200 mL of anhydrous dimethylformamide. To this solution was added dropwise isobutyl chloroformate (16.4 mL). The reaction mixture was stirred for 30 minutes and hydroxylamine hydrochloride (17.6 g) was added followed by dropwise addition of 35 mL of TEA at 0-5° C. The reaction was allowed to warm to room temperature and stirred overnight. The reaction was quenched with 250 mL of 1% (by weight) citric acid solution and 50 mL of 5% (by weight) citric acid solution and then extracted with 200 mL of methylene chloride twice and 200 mL of ether once. The solvents were removed under vacuum. The residue was triturated with 125 mL of water, filtered, washed with 25 mL of water and dried under vacuum to give a tan solid. The crude product was chromatographed on a Biotage 75S column and eluted with methylene chloride:acetonitrile (80:20). The fractions containing the desired product were combined and the solvent was removed under vacuum to yield 4.1 g of cinnamoylhydroxamic acid. ¹H NMR (DMSO-d₆, 300 MHz), δ(ppm) 7.48 (m, 6H), 6.49 (d, 1H).

### EXAMPLE 13

### Synthesis of N-methyl-cinnamoylhydroxamic acid

A solution of cinnamoyl chloride (5 g) in 50 mL of methylene chloride was added dropwise to a solution of N-methylhydroxylamine hydrochloride (5 g) and 12 mL of 40% sodium hydroxide in 50 mL of water cooled to 0-5° C. The reaction mixture was stirred for two hours. The aqueous layer was acidified with concentrated hydrochloric acid. The precipitate was collected by filtration and dried under vacuum to afford 2.8 g of the desired N-methyl-cinnamoylhydroxamic acid as a white solid. ¹H NMR (DMSO-d₆, 300 MHz), δ(ppm) 7.66 (d, 2H), 7.53 (d, 1H), 7.42 (m, 3H), 7.26 (d, 1H), 3.22 (s, 3H).

### EXAMPLE 14

### Synthesis of 5-phenyl-2,4-pentadienoylhydroxamic acid

Triethylamine (TEA, 29 mL) was added to a cooled (0-5° C.) solution of 5-phenyl-2,4-pentadienoic acid (29.0 g) in 300 mL of anhydrous dimethylformamide. To this solution was added dropwise isobutyl chloroformate (27.0 mL). The reaction mixture was stirred for 15 minutes and hydroxylamine hydrochloride (28.92 g) was added followed by dropwise addition of 58 mL of TEA over a period of 60 minutes at 0-5° C. The reaction was allowed to warm to room temperature and stirred overnight. The reaction was then poured into 450 mL of a 1% (by weight) solution of citric acid and then extracted with 200 mL of methylene chloride twice and 500 mL of ether once. The solvents were removed under vacuum to give an oil. The crude oil was crystallized with 200 mL of hot acetonitrile to give a tan solid. The tan solid was recrystallized from 60 mL of hot acetonitrile to afford 12.5 g of the desired 5-phenyl-2,4-pentadienoylhydroxamic acid. ¹H NMR (DMSO-d₆, 300 MHz), δ(ppm) 7.56 (d, 2H), 7.31 (m, 4H), 7.03 (m, 2H), 6.05 (s, 1H).

### EXAMPLE 15

### Synthesis of N-methyl-5-phenyl-2,4-pentadienoylhydroxamic acid

5-Phenyl-2,4-pentadienoic acid (6 g) and oxalyl chloride (6.1 mL) were dissolved in 50 mL of methylene chloride and 0.2 mL of dimethylformamide was added. The reaction was stirred for three hours, concentrated under vacuum and then co-evaporated with 100 mL of chloroform to remove oxalyl chloride. The crude 5-phenyl-2,4-pentadienoic acid chloride was used in the next step without further purification.

5-Phenyl-2,4-pentadienoic acid chloride was dissolved in 50 mL of methylene chloride and added to a solution of 13.8 mL of 40% sodium hydroxide in 50 mL of water at 0-5° C. The resulting solution was stirred for two hours and then acidified to a pH of 4 with concentrated hydrochloric acid. The precipitate was collected by filtration and dried under vacuum to afford 4.2 g of N-methyl-5-phenyl-2,4-pentadienoylhydroxamic acid. ¹H NMR (DMSO-d₆, 300 MHz), δ(ppm) 7.57 (d, 2H), 7.35 (m, 4H), 7.19 (m, 1H), 6.99 (d, 1H), 6.82 (d, 1H), 3.21 (s, 3H).

### EXAMPLE 16

### Synthesis of 3-methyl-5-phenyl-2,4-pentadienoylhydroxamic acid

Triethylamine (TEA, 1.8 mL) was added to a cooled (0-5° C.) solution of 3-methyl-5-phenyl-2,4-pentadienoic acid (2.0 g) in 20 mL of anhydrous dimethylformamide. To this solution was added dropwise isobutyl chloroformate (1.7 mL) over a period of 15 minutes. The reaction mixture was stirred for 30 minutes and hydroxylamine hydrochloride (1.85 g) was added followed by dropwise addition of 3.7 mL of TEA over a period of 35 minutes at 0-5° C. The reaction was allowed to warm to room temperature and stirred overnight. To the stirred reaction mixture at room temperature was added 20 mL of a 1% (by weight) solution of citric acid followed by 75 mL of water. The mixture was stirred for 30 minutes and then filtered. The filtered cake was washed with 30 mL of water and dried in vacuum to afford 1.49 g of the desired 3-methyl-5-phenyl-2,4-pentadienoylhydroxamic acid in 69% yield. ¹H NMR (DMSO-d₆, 300 MHz), δ(ppm) 7.55 (d, 2H), 7.30 (m, 3H), 6.89 (broad s, 2H), 5.83 (s, 1H), 2.38 (s, 3H).

### EXAMPLE 17

### Synthesis of 4-methyl-5-phenyl-2,4-pentadienoylhydroxamic acid

Triethylamine (TEA, 6.5 mL) was added to a cooled (0-5° C.) solution of 4-methyl-5-phenyl-2,4-pentadienoic acid (7.0 g) in 75 mL of anhydrous dimethylformamide. To this solution was added dropwise isobutyl chloroformate (6.0 mL) over a period of 60 minutes. The reaction mixture was stirred for 15 minutes and hydroxylamine hydrochloride (6.5 g) was added followed by dropwise addition of 13 mL of TEA over a period of 60 minutes at 0-5° C. The reaction was allowed to warm to room temperature and stirred overnight. To the stirred reaction mixture at room temperature was added 130 mL of a 1% (by weight) solution of citric acid followed by 50 mL of water. The mixture was stirred for 30 minutes and then filtered. The filtered cake was recrystallized from hot acetonitrile to afford 4.4 g of the desired 4-methyl-5-phenyl-2,4-pentadienoylhydroxamic acid. ¹H NMR (DMSO-d₆, 300 MHz), δ(ppm) 7.37 (m, 6H), 6.91 (s, 1H), 6.02 (d, 1H), 1.99 (s, 3H).

### EXAMPLE 18

### Synthesis of 4-chloro-5-phenyl-2,4-pentadienoylhydroxamic acid

Triethylamine (TEA, 2.5 mL) was added to a cooled (0-5° C.) solution of 4-chloro-5-phenyl-2,4-pentadienoic acid (3.0 g) in 30 mL of anhydrous dimethylformamide. To this solution was added dropwise isobutyl chloroformate (2.3 mL) over a period of 15 minutes. The reaction mixture was stirred for 30 minutes and hydroxylamine hydrochloride (2.5 g) was added followed by dropwise addition of 5.0 mL of TEA over a period of 60 minutes at 0-5° C. The reaction was allowed to warm to room temperature and stirred overnight. The reaction was then quenched with 30 mL of a 1% (by weight) solution of citric acid followed by 115 mL of water. The mixture was stirred for 30 minutes and then filtered. The filtered cake was washed with 100 mL of water and dried under vacuum. The crude material was recrystallized from 20 mL of hot acetonitrile twice to yield 1.46 g of the desired 4-chloro-5-phenyl-2,4-pentadienoylhydroxamic acid as a solid. ¹H NMR (DMSO-d₆, 300 MHz), δ(ppm) 7.75 (d, 2H), 7.40 (m, 5H), 6.31 (d, 1H).

### EXAMPLE 19

### Synthesis of 5-phenyl-2-ene-4-pentynoylhydroxamic acid

Triethylamine (TEA, 1.1 mL) was added to a cooled (0-5° C.) solution of 5-phenyl-2-ene-4-pentynoic acid (1.1 g) in 13 mL of anhydrous dimethylformamide. To this solution was added dropwise isobutyl chloroformate (1.0 mL). The reaction mixture was stirred for 30 minutes and hydroxylamine hydrochloride (1.1 g) was added followed by dropwise addition of 2.2 mL of TEA at 0-5° C. The reaction was allowed to warm to room temperature and stirred overnight. The reaction was quenched with 15 mL of a 1% (by weight) solution of citric acid and extracted with 30 mL of methylene chloride twice. The combined organic layer was dried over anhydrous sodium sulfate. The solvents were removed under vacuum to give an oil which in turn was triturated with 10 mL of chloroform. The solid was collected by filtration to yield 0.63 g of the desired 5-phenyl-2-ene-4-pentynoylhydroxamic acid as a white powder. ¹H NMR (DMSO-d₆, 300 MHz), δ(ppm) 7.48 (m, 5H), 6.76 (d, 1H), 6.35 (d, 1H).

### EXAMPLE 20

### Synthesis of 5-(p-dimethylaminophenyl)-2,4-pentadienoylhydroxamic acid

Triethylamine (TEA, 0.8 mL) was added to a cooled (0-5° C.) solution of 5-(p-dimethylaminophenyl)-2,4-pentadienoic acid (1.0 g) in 10 mL of anhydrous dimethylformamide. To this solution was added dropwise isobutyl chloroformate (0.7 mL). The reaction mixture was stirred for 60 minutes and hydroxylamine hydrochloride (0.8 g) was added followed by dropwise addition of 1.6 mL of TEA at 0-5° C. The reaction was allowed to warm to room temperature and stirred overnight. The reaction was quenched with 15 mL of water. The solid was filtered and dried under vacuum to yield 0.75 g of the desired 5-(p-dimethylaminophenyl)-2,4-pentadienoylhydroxamic acid. ¹H NMR (DMSO-d₆, 300 MHz), δ(ppm) 7.33 (m, 3H), 6.86 (m, 2H), 6.70 (d, 2H), 5.84 (d, 1H), 2.99 (s, 6H).

### EXAMPLE 21

### Synthesis of 5-(2-furyl)-2,4-pentadienoylhydroxamic acid

Triethylamine (TEA, 2.1 mL) was added to a cooled (0-5° C.) solution of 5-(2-furyl)-2,4-pentadienoic acid (2.0 g) in 15 mL of anhydrous dimethylformamide. To this solution was added dropwise isobutyl chloroformate (2.0 mL) over a period of 30 minutes. The reaction mixture was stirred for 30 minutes and hydroxylamine hydrochloride (2.15 g) was added followed by dropwise addition of 4.2 mL of TEA over a period of 60 minutes at 0-5° C. The reaction was allowed to warm to room temperature and stirred overnight. To the stirred reaction mixture at room temperature was added 12 mL of a 1% (by weight) solution of citric acid followed by 46 mL of water. The mixture was stirred for 30 minutes and then filtered. The filtered cake was washed with 30 mL of water and dried in vacuum to afford 1.3 g of the desired 5-(2-furyl)-2,4-pentadienoylhydroxamic acid. ¹H NMR (DMSO-d₆, 300 MHz), δ(ppm) 7.73 (broad s, 1H), 7.22 (m, 1H), 6.71 (m, 4H), 6.01 (d, 1H).

### EXAMPLE 22

### Synthesis of 6-phenyl-3,5-hexadienoylhydroxamic acid

Triethylamine (TEA, 1.75 mL) was added to a cooled (0-5° C.) solution of 6-phenyl-3,5-hexadienoic acid (2.0 g) in 30 mL of anhydrous dimethylformamide. To this solution was added dropwise isobutyl chloroformate (1.62 mL) over a period of 15 minutes. The reaction mixture was stirred for 15 minutes and hydroxylamine hydrochloride (1.74 g) was added followed by dropwise addition of 3.5 mL of TEA at 0-5° C. The reaction was allowed to warm to room temperature and stirred overnight. The reaction was then poured into 20 mL of 1% (by weight) aqueous citric acid solution and extracted with 20 mL of methylene chloride twice and ether once. The combined organic layer was dried over anhydrous sodium sulfate and concentrated under vacuum to give a dark red oil. The crude oil was crystallized with 10 mL of hot acetonitrile. The solid was collected by filtration and then purified on a Biotage 40S silica gel column using methylene chloride:ether (95:5) as an eluent. The fractions containing the desired product were combined and the solvent was removed to give 40 mg of 6-phenyl-3,5-hexadienoylhydroxamic acid as a tan solid (2.1%). ¹H NMR (DMSO-d₆, 300 MHz), δ(ppm) 7.34 (m, 5H), 6.91 (m, 1H), 6.55 (d, 1H), 6.30 (m, 1H), 5.89 (m, 1H), 3.36 (d, 2H).

### EXAMPLE 23

### Synthesis of N-methyl-6-phenyl-3,5-hexadienoylhydroxamic acid

6-Phenyl-3,5-hexadienoic acid (1 g) was dissolved in 10 mL of tetrahydrofuran (THF) and treated with 0.9 g of 1,1′-carbonyldiimidazole. The reaction was stirred for 30 minutes. N-methylhydroxylamine hydrochloride (0.44 g) was neutralized with 0.29 g of sodium methoxide in 10 mL of THF and 5 mL of methanol and then filtered to remove the sodium chloride. N-methylhydroxylamine was then added to the reaction mixture and stirred overnight. The resulting mixture was partitioned between 25 mL of water and 50 mL of ethyl acetate. The ethyl acetate layer was washed with 25 mL each of 5% hydrochloric acid, saturated sodium bicarbonate and brine, dried over sodium sulfate and concentrated under vacuum to afford 0.9 g of a viscous yellow oil. The crude product was chromatographed on a Biotage 40S silica gel column and eluted with ethyl acetate:hexane (1:1). The fractions containing the desired product were combined and the solvent was removed under vacuum to yield 0.17 g of N-methyl-6-phenyl-3,5-hexadienoylhydroxamic acid. ¹H NMR (CDCl₃, 300 MHz), δ(ppm) 7.38 (m, 5H), 6.80 (m, 1H), 6.60 (m, 1H), 6.35 (m, 1H), 5.89 (m, 1H), 3.24 (m, 2H), 2.92 (s, 3H).

### EXAMPLE 24

### Synthesis of 7-phenyl-2,4,6-heptatrienoic acid

To a cooled (0-55° C.) 927 mL of 1 M solution of phenyl magnesium bromide in tetrahydofuran was added dropwise a solution of crotonaldehyde (65.0 g) in 130 mL of anhydrous ether over a period of 2 hours and 45 minutes. The reaction was stirred for an additional 45 minutes and then warmed to room temperature. After four more hours of stirring, saturated ammonium chloride aqueous solution (750 mL) was added to the reaction. The mixture was extracted with 750 mL of ether twice. The combined extract was dried over anhydrous potassium carbonate and filtered. The solvent was evaporated to give 135.88 g (99.9%) of the desired 1-phenyl-2-buten-1-ol as an oil which was used in the next step without further purification.

1-Phenyl-2-buten-1-ol (135.88 g) was dissolved in 2300 mL of dioxane and treated with 2750 mL of dilute hydrochloric acid (2.3 mL of concentrated hydrochloric acid in 2750 mL of water) at room temperature. The mixture was stirred overnight and then poured into 4333 mL of ether and neutralized with 2265 mL of saturated aqueous sodium bicarbonate. The aqueous phase was extracted with 1970 mL of ether. The combined extract was dried over anhydrous potassium carbonate. Evaporation of the solvent followed by Kugelrohr distillation at 30° C. for 30 minutes afforded 131.73 g (96.8%) of the desired 4-phenyl-3-buten-2-ol as an oil which was used in the next step without further purification.

Dimethylformamide (DMF, anhydrous, 14 mL) was cooled to 0-5° C. and phosphorus oxychloride (8.2 mL) was added dropwise over a period of 40 minutes. The resulting solution was added dropwise to a cooled (0-5° C.) solution of 4-phenyl-3-buten-2-ol (10 g) in 32 mL of anhydrous DMF over a period of an hour. The reaction mixture was warmed to room temperature over a 35-minute period and then gradually heated up to 80° C. over a period of 45 minutes. The reaction was stirred at 80° C. for three hours and then cooled to 0-5° C. To the cooled reaction solution was added dropwise a solution of sodium acetate (40 g) in deionized water (100 mL) over a period of one hour. The mixture was then reheated to 80° C., stirred at 80° C. for an additional 10 minutes, cooled down to room temperature and extracted with ether (100 mL) twice. The combined extract was washed with brine (100 mL), dried over anhydrous sodium sulfate, filtered and concentrated under vacuum to yield 8.78 g of the desired 5-phenyl-2,4-pentadienal as a liquid which was used in the next step without further purification. ¹H NMR (CDCl₃, 300 MHz), δ(ppm) 7.51 (m, 2H), 7.37 (m, 3H), 7.26 (m, 1H), 7.01 (m, 2H), 6.26 (m, 1H).

Butyllithium (12.8 mL of 2.5 N solution) was added to 65 mL of anhydrous tetrahydrofuran (THF) at −65° C. A solution of diethylphosphonoacetic acid (2.92 g) in 25 mL of anhydrous THF was added dropwise to the stirred solution at −65° C. The resulting solution was stirred at −65° C. for an additional 30 minutes and then a solution of 5-phenyl-2,4-pentadienal (2.4 g) in 15 mL of anhydrous THF was added to the reaction at −65° C. The reaction was stirred for one hour, allowed to warm to room temperature and then stirred overnight. To the reaction was added 30 mL of water, acidified with 5% hydrochloric acid (14 mL) to a pH of 4.7 and then added an additional 20 mL of water. The aqueous layer was extracted with 10 mL of ether twice and with 10 mL of ethyl acetate once. The combined organic extract was dried over anhydrous sodium sulfate, filtered and concentrated under vacuum. The crude material was dissolved in 50 mL of hot methanol and then refrigerated overnight. The crystals formed were filtered and dried under vacuum to afford 2.4 g of the desired 7-phenyl-2,4,6-heptatrienoic acid. ¹H NMR (DMSO-d₆, 300 MHz), δ(ppm) 7.52 (m, 2H), 7.33 (m, 4H), 7.06 (m, 1H), 6.86 (m, 2H), 6.58 (m, 1H), 5.95 (d, 1H).

### EXAMPLE 25

### Synthesis of 4-cyclohexylbutyroylhydroxamic acid

To a solution of hydroxylamine hydrochloride (7.3 g) in 50 mL of methanol was added 24 mL of sodium methoxide (25% wt.) dropwise at room temperature over a period of 45 minutes. To this solution was added methyl 4-cyclohexylbutyrate in 50 mL of methanol at room temperature followed by 12 mL of sodium methoxide (25% wt.) dropwise over a period of 60 minutes. The resulting mixture was stirred at room temperature overnight. The reaction was then poured into 120 mL of water and acidified to a pH of 4 with 45 mL of glacial acetic acid. Methanol was removed under vacuum. The solid formed was filtered and dried over phosphorus pentoxide to afford 8.53 g of the desired 4-cyclohexylbutyroyl-hydroxamic acid. ¹H NMR (DMSO-d₆, 300 MHz), δ(ppm) 3.38 (m, 2H), 1.91 (t, 2H), 1.68 (m, 4H), 1.50 (m, 2H), 1.16 (m, 5H), 0.84 (m, 2H).

### EXAMPLE 26

### Synthesis of S-benzylthioglycoloylhydroxamic acid

S-benzylthioglycolic acid (12.0 g) was dissolved in 250 mL of methanol and sparged with hydrogen chloride gas at room temperature for 20 minutes. The solvent was then removed under vacuum. Methyl S-benzylthioglycolate obtained was used in the next step without further purification.

To a solution of hydroxylamine hydrochloride (9.2 g) in 60 mL of methanol was added 30 mL of sodium methoxide (25% wt.) dropwise at room temperature over a period of 30 minutes. To this solution was added methyl S-benzylthioglycolate in 50 mL of methanol at room temperature followed by 15 mL of sodium methoxide (25% wt.) dropwise over a period of 60 minutes. The resulting mixture was stirred at room temperature overnight. The reaction was then poured into 150 mL of water and acidified to a pH of 4 with 55 mL of glacial acetic acid. Methanol was removed under vacuum. The solid formed was filtered and dried over phosphorus pentoxide to afford 8.57 g of the desired S-benzylthioglycoloyl-hydroxamic acid. ¹H NMR (DMSO-d₆, 300 MHz), δ(ppm) 7.29 (m, 5H), 3.84 (s, 2H), 2.93 (s, 2H).

### EXAMPLE 27

### Synthesis of 5-phenylpentanoloylhydroxamic acid

5-Phenylpentanoic acid (10.0 g) was dissolved in 250 mL of methanol and sparged with hydrogen chloride gas at room temperature for 15 minutes. The solvent was then removed under vacuum. Methyl 5-phenylpentanoate obtained was used in the next step without further purification.

To a solution of hydroxylamine hydrochloride (7.8 g) in 50 mL of methanol was added 26 mL of sodium methoxide (25% wt.) dropwise at room temperature over a period of 45 minutes. To this solution was added methyl 5-phenylpentanoate in 50 mL of methanol at room temperature followed by 15 mL of sodium methoxide (25% wt.) dropwise over a period of 60 minutes. The resulting mixture was stirred at room temperature overnight. The reaction was then poured into 150 mL of water and acidified to a pH of 4 with 40 mL of glacial acetic acid. The solvents were removed under vacuum to give a yellow oil. The yellow oil was placed on a Biotage 40M silica gel column and eluted with methylene chloride:ethanol (95:5). The fractions containing the desired product as indicated by the NMR were combined. The solvents were removed under vacuum to afford 8.30 g of the desired 5-phenylpentanoylhydroxamic acid. ¹H NMR (DMSO-d₆, 300 MHz), δ(ppm) 7.22 (m, 5H), 3.42 (s, 3H), 2.55 (t, 2H), 1.98 (t, 2H), 1.52 (m, 4H).

### EXAMPLE 28

### Stabilization of p53 acetylation by 7-phenyl-2,4,6-hepta-trienoic hydroxamic acid

In addition to increasing the level of histone acetylation, 7-phenyl-2,4,6-heptatrienoic hydroxamic acid also stabilizes the acetylation of p53 at amino acids Lys373 and Lys382 but not Lys320. 7-phenyl-2,4,6-heptatrienoic hydroxamic acid also increases the levels of total p53 in LNCaP cells (human prostate cancer cells). Activated, acetylated p53 induced p53-dependent increase in p21 levels, leading to cell cycle arrest, primarily at G2/M interface. In addition, 7-phenyl-2,4,6-heptatrienoic hydroxamic acid also increased the steady state level of cytosolic Bax, and induced Bax mitochondrial translocation and cleavage which in turn leads to induction of selective degradation of HDAC2.

Comparison of the effects of 7-phenyl-2,4,6-heptatrienoic hydroxamic acid and trichostatin (TSA) has shown that while TSA induced p21 and cell cycle arrest, it did not alter Bax levels nor did it affect Bax translocation and cleavage.

### EXAMPLE 29

### Inhibition of HDAC1 and HDAC2 by 7-phenyl-2,4,6-heptatrienoic hydroxamic acid

To determine whether the differential effects are cell line specific or whether 7-phenyl-2,4,6-heptatrienoic hydroxamic acid and TSA target different HDACs, the activity of both compounds was compared in PC-3 cells. PC-3 cells are p53−/− and do not express HDAC2. The p53 dependent activation of Bax was absent in PC-3 cells after treatment with either 7-phenyl-2,4,6-heptatrienoic hydroxamic acid or TSA. However, p53 independent p21 activation was observed and this was probably due to the inhibition of HDAC1. These results indicate that HDAC1 and HDAC2 are important regulators of p53 acetylation, leading to stabilization of acetylated p53 and downstream activation of p21 and Bax.

### Other Embodiments

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of inhibiting HDAC2 in a cell comprising contacting the cell with an amount of a hydroxamic acid compound effective to inhibit deacetylation activity of HDAC2.

2. The method of claim 1, wherein the hydroxamic acid compound if of formula (I), the compound having the following formula 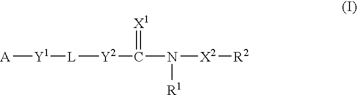
wherein
A is a cyclic moiety selected from the group consisting of C₃₋₁₄ cycloalkyl, 3-14 membered heterocycloalkyl, C₄₋₁₄ cycloalkenyl, 3-14 membered heterocycloalkenyl, monocyclic aryl, or monocyclic heteroaryl; the cyclic moiety being optionally substituted with alkyl, alkenyl, alkynyl, alkoxy, hydroxyl, hydroxylalkyl, halo, haloalkyl, amino, alkylcarbonyloxy, alkyloxycarbonyl, alkylcarbonyl, alkylsulfonylamino, aminosulfonyl, or alkylsulfonyl;
each of X¹ and X², independently, is O or S;
Y¹ is —CH₂—, —O—, —S—, —N(R^{a})—, —N(R^{a})—C(O)—O—, —O—C(O)—N(R^{a})—, —N(R^{a})—C(O)—N(R^{b})—, —C(O)—O—, —O—C(O)—O—, —N(R^{a})—C(O)—, —C(O)—N(R^{a})—, or a bond; each of R^{a} and R^{b}, independently, being hydrogen, alkyl, alkenyl, alkynyl, alkoxy, hydroxylalkyl, hydroxyl, or haloalkyl;
Y² is a bond;
L is an unsaturated straight C₄₋₁₂ hydrocarbon chain containing at least two double bonds, at least one triple bond, or at least one double bond and one triple bond, or a saturated C₄₋₈ hydrocarbon chain; the hydrocarbon chain being optionally substituted with C₁₋₄ alkyl, C₂₋₄ alkenyl, C₂₋₄ alkynyl, C₁₋₄ alkoxy, hydroxyl, halo, carboxyl, amino, nitro, cyano, C₃₋₆ cycloalkyl, 3-6 membered heterocycloalkyl, monocyclic aryl, 5-6 membered heteroaryl, C₁₋₄ alkylcarbonyloxy, C₁₋₄ alkyloxycarbonyl, C₁₋₄ alkylcarbonyl, oxo or formyl; and further being optionally interrupted by —O—, —N(R^{g})—, —N(R^{g})—C(O)—O—, —O—C(O)—N(R^{g})—, —N(R^{g})—C(O)—N(R^{h})—, —O—C(O)—, —C(O)—O—, or —O—C(O)—O—; each of R^{g} and R^{h,} independently, being hydrogen, alkyl, alkenyl, alkynyl, alkoxy, hydroxylalkyl, hydroxyl, or haloalkyl;
R¹ is hydrogen, alkyl, alkenyl, alkynyl, alkoxy, hydroxylalkyl, hydroxyl, haloalkyl, or an amino protecting group; and
R² is hydrogen, alkyl, hydroxylalkyl, haloalkyl, or a hydroxyl protecting group;
or a salt thereof.

3. The method of claim 2, wherein the carbon bonded to Y² is unsaturated, and provided that when L is a C₄₋₅ hydrocarbon chain and contains two double bonds, Y¹ is not CH₂.

4. The method of claim 2, wherein R¹ is hydrogen.

5. The method of claim 2, wherein R² is hydrogen.

6. The method of claim 2, wherein each of R¹ and R² is hydrogen.

7. The method of claim 2, wherein X¹ is O.

8. The method of claim 2, wherein X² is O.

9. The method of claim 2, wherein each of X¹ and X² is O.

10. The method of claim 2, wherein Y¹ is —CH₂—, —O—, —N(R^{a})—, or a bond.

11. The method of claim 2, wherein Y¹ is a bond.

12. The method of claim 2, wherein L is an unsaturated straight C₄₋₁₀ hydrocarbon chain optionally substituted with C₁₋₄ alkyl, C₂₋₄ alkenyl, C₂₋₄ alkynyl, C,₄ alkoxy, or amino.

13. The method of claim 2, wherein L is an unsaturated straight C₅₋₈ hydrocarbon chain optionally substituted with C₁₋₄ alkyl, C₂₋₄ alkenyl, C₂₋₄ alkynyl, C₁₋₄ alkoxy, or amino.

14. The method of claim 2, wherein L is an unsubstituted unsaturated straight C₄₋₆ hydrocarbon chain.

15. The method of claim 2, wherein L is an unsubstituted unsaturated straight C₅ hydrocarbon chain.

16. The method of claim 2, wherein L is an unsubstituted unsaturated straight C₆ hydrocarbon chain.

17. The method of claim 2, wherein L is an unsaturated straight C₄₋₁₀ hydrocarbon chain containing 2-5 double bonds optionally substituted with C₁₋₄ alkyl, C₂₋₄ alkenyl, C₂₋₄ alkynyl, or C₁₋₄ alkoxy.

18. The method of claim 2, wherein L is an unsaturated straight C₄₋₈ hydrocarbon chain containing 2-5 double bonds optionally substituted with C₁₋₄ alkyl, C₂₋₄ alkenyl, C₂₋₄ alkynyl, or C₁₋₄ alkoxy.

19. The method of claim 2, wherein L is —(CH═CH)ₘ— where m is 2 or 3, L being optionally substituted with C₁₋₄ alkyl, C₂₋₄ alkenyl, C₂₋₄ alkynyl, or C₁₋₄ alkoxy.

20. The method of claim 2, wherein L is an unsaturated straight C₄₋₁₀ hydrocarbon chain containing 1-2 double bonds and 1-2 triple bonds, the hydrocarbon chain being optionally substituted with C₁₋₄ alkyl, C₂₋₄ alkenyl, C₂₋₄ alkynyl, or C₁₋₄ alkoxy.

21. The method of claim 2, wherein L is an unsaturated straight C₄₋₈ hydrocarbon chain containing 1-2 double bonds and 1-2 triple bonds, the hydrocarbon chain being optionally substituted with C₁₋₄ alkyl, C₂₋₄ alkenyl, C₂₋₄ alkynyl, or C₁₋₄ alkoxy.

22. The method of claim 2, wherein L is —C≡C—(CH═CH)ₙ— where n is 1 or 2, L being optionally substituted with C₁₋₄ alkyl, C₂₋₄ alkenyl, C₂₋₄ alkynyl, or C₁₋₄ alkoxy.

23. The method of claim 2, wherein A is phenyl.

24. The method of claim 2, wherein A is phenyl optionally substituted with alkyl, alkenyl, alkynyl, alkoxy, hydroxylalkyl, or amino.

25. The method of claim 24, wherein L is an unsaturated straight C₄₋₆ hydrocarbon chain.

26. The method of claim 25, wherein L is a saturated straight C₆ hydrocarbon chain.

27. The-method of claim 26, wherein each of R¹ and R² is hydrogen.

28. The method of claim 27, wherein each of X¹ and X² is O.

29. The method of claim 28, wherein Y¹ is —CH₂—, —O—, —N(R^{a})—, or a bond.

30. The method of claim 24, wherein L is an unsaturated straight C₄₋₈ hydrocarbon chain containing 2-5 double bonds; the hydrocarbon chain being optionally substituted with C₁₋₄ alkyl, C₂₋₄ alkenyl, C₂₋₄ alkynyl, or C₁₋₄ alkoxy.

31. The method of claim 30, wherein L is —(CH═CH)ₘ—, where m is 2 or 3.

32. The method of claim 31, wherein each of R¹ and R² is hydrogen.

33. The method of claim 32, wherein each of X¹ and X² is O.

34. The method of claim 33, wherein Y¹ is —CH₂—, —O—, —N(R^{a})—, or a bond.

35. The method of claim 24, wherein L is an unsaturated straight C₄₋₈ hydrocarbon chain containing 1-2 double bonds and 1-2 triple bonds; the hydrocarbon chain being optionally substituted with C₁₋₄ alkyl, C₂₋₄ alkenyl, C₂₋₄ alkynyl, or C₁₋₄ alkoxy.

36. The method of claim 35, wherein L is —C≡C—(CH═CH)ₙ—, where n is 1 or 2.

37. The method of claim 34, wherein each of R¹ and R² is hydrogen.

38. The method of claim 36, wherein each of X¹ and X² is O.

39. The method of claim 38, wherein Y¹ is —CH₂—, —O—, —N(R^{a})—, or a bond.

40. The method of claim 1, wherein the compound is 5-phenyl-2,4-pentadienoyl hydroxamic acid, N-methyl-5-phenyl-2,4-pentadienoyl hydroxamic acid, 3-methyl-5-phenyl-2,4-pentadienoyl hydroxamic acid, 4-methyl-5-phenyl-2,4-pentadienoyl hydroxamic acid, 4-chloro-5-phenyl-2,4-pentadienoyl hydroxamic acid, 5-(4-dimethylaminophenyl)-2,4-pentadienoyl hydroxamic acid, 5-phenyl-2-en-4-yn-pentanoyl hydroxamic acid, N-methyl-6-phenyl-3,5-hexadienoyl hydroxamic acid, potassium 2-oxo-6-phenyl-3,5-hexadienoate, potassium 2-oxo-8-phenyl-3,5,7-octatrienoate, or 7-phenyl-2,4,6-hepta-trienoylhydroxamic acid.

41. The method of claim 1, wherein the compound is 7-phenyl-2,4,6-heptatrienoylhydroxamic acid.

42. The method of claim 1, wherein the compound further inhibits the deacetylation of p53 in the cell.

43. The method of claim 1, wherein the compound further increases the levels of p21 in the cell.

44. The method of claim 1, wherein the compound further increases levels of Bax in the cell.

45. The method of claim 1, wherein the compound further induces cell cycle arrest in the cell.

46. The method of claim 1, wherein the compound further induces apoptosis in the cell.

47. The method of claim 1, wherein the cell is contacted with the compound in vivo.

48. The method of claim 1, wherein the cell is contacted with the compound in vitro.

49. A method of inhibiting HDAC1 in a cell comprising contacting the cell with an amount of a hydroxamic acid compound effective to inhibit deacetylation activity of HDAC1.

50. The method of claim 49, wherein the hydroxamic acid compound is of formula (I), the compound having the following formula 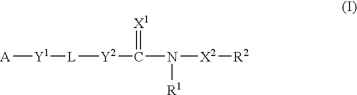
wherein
A is a cyclic moiety selected from the group consisting of C₃₋₁₄ cycloalkyl, 3-14 membered heterocycloalkyl, C₄₋₁₄ cycloalkenyl, 3-14 membered heterocycloalkenyl, monocyclic aryl, or monocyclic heteroaryl; the cyclic moiety being optionally substituted with alkyl, alkenyl, alkynyl, alkoxy, hydroxyl, hydroxylalkyl, halo, haloalkyl, amino, alkylcarbonyloxy, alkyloxycarbonyl, alkylcarbonyl, alkylsulfonylamino, aminosulfonyl, or alkylsulfonyl;
each of X¹ and X², independently, is O or S;
Y¹ is —CH₂—, —O—, —S—, —N(R^{a})—, —N(R^{a})—C(O)—O—, —O—C(O)—N(R^{a})—, —N(R^{a})—C(O)—N(R^{b})—, —C(O)—O—, —O—C(O)—O—, —N(R^{a})—C(O)—, —C(O)—N(R^{a})—, or a bond; each of R^{a} and R^{b}, independently, being hydrogen, alkyl, alkenyl, alkynyl, alkoxy, hydroxylalkyl, hydroxyl, or haloalkyl;
Y² is a bond;
L is an unsaturated straight C₄₋₁₂ hydrocarbon chain containing at least two double bonds, at least one triple bond, or at least one double bond and one triple bond, or a saturated C₄₋₈ hydrocarbon chain; the hydrocarbon chain being optionally substituted with C₁₋₄ alkyl, C₂₋₄ alkenyl, C₂₋₄ alkynyl, C₁₋₄ alkoxy, hydroxyl, halo, carboxyl, amino, nitro, cyano, C₃₋₆ cycloalkyl, 3-6 membered heterocycloalkyl, monocyclic aryl, 5-6 membered heteroaryl, C₁₋₄ alkylcarbonyloxy, C₁₋₄ alkyloxycarbonyl, C₁₋₄ alkylcarbonyl, oxo or formyl; and further being optionally interrupted by —O—, —N(R^{g})—, —N(R^{g})—C(O)—O—, —O—C(O)—N(R^{g})—, —N(R^{g})—C(O)—N(R^{h})—, —O—C(O)—, —C(O)—O—, or —O—C(O)—O—; each of R^{g} and R^{h}, independently, being hydrogen, alkyl, alkenyl, alkynyl, alkoxy, hydroxylalkyl, hydroxyl, or haloalkyl;
R¹ is hydrogen, alkyl, alkenyl, alkynyl, alkoxy, hydroxylalkyl, hydroxyl, haloalkyl, or an amino protecting group; and
R² is hydrogen, alkyl, hydroxylalkyl, haloalkyl, or a hydroxyl protecting group;
or a salt thereof.

51. The method of claim 50, wherein the carbon bonded to Y² is unsaturated, and provided that when L is a C₄₋₅ hydrocarbon chain and contains two double bonds, Y¹ is not CH₂.

52. The method of claim 49, wherein the compound is 7-phenyl-2,4,6-heptatrienoylhydroxamic acid.

53. The method of claim 49, wherein the compound further increases the levels of p21 in the cell.

54. The method of claim 49, wherein the compound further induces cell cycle arrest in the cell.

55. The method of claim 49, wherein the cell is contacted with the compound in vivo.

56. The method of claim 49, wherein the cell is contacted with the compound in vitro.

57. A method of treating hormone-refractory metastatic prostate cancer in a mammal comprising administering to the mammal an effective amount of a compound (I); the compound having the following formula 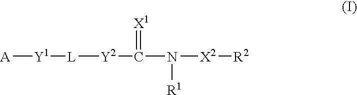
wherein
A is a cyclic moiety selected from the group consisting of C₃₋₁₄ cycloalkyl, 3-14 membered heterocycloalkyl, C₄₋₁₄ cycloalkenyl, 3-14 membered heterocycloalkenyl, monocyclic aryl, or monocyclic heteroaryl; the cyclic moiety being optionally substituted with alkyl, alkenyl, alkynyl, alkoxy, hydroxyl, hydroxylalkyl, halo, haloalkyl, amino, alkylcarbonyloxy, alkyloxycarbonyl, alkylcarbonyl, alkylsulfonylamino, aminosulfonyl, or alkylsulfonyl;
each of X¹ and X², independently, is O or S;
Y¹ is —CH₂—, —O—, —S—, —N(R^{a})—, —N(R^{a})—C(O)—O—, —O—C(O)—N(R^{a})—, —N(R^{a})—C(O)—N(R^{b})—, —C(O)—O—, —O—(O)—O—, —N(R^{a})—C(O)—, —C(O)—N(R^{a})—, or a bond; each of R^{a} and R^{b}, independently, being hydrogen, alkyl, alkenyl, alkynyl, alkoxy, hydroxylalkyl, hydroxyl, or haloalkyl;
Y² is a bond;
L is an unsaturated straight C₄₋₁₂ hydrocarbon chain containing at least two double bonds, at least one triple bond, or at least one double bond and one triple bond, or a saturated C₄₋₈ hydrocarbon chain; the hydrocarbon chain being optionally substituted with C₁₋₄ alkyl, C₂₋₄ alkenyl, C₂₋₄ alkynyl, C₁₋₄ alkoxy, hydroxyl, halo, carboxyl, amino, nitro, cyano, C₃₋₆ cycloalkyl, 3-6 membered heterocycloalkyl, monocyclic aryl, 5-6 membered heteroaryl, C₁₋₄ alkylcarbonyloxy, C₁₋₄ alkyloxycarbonyl, C₁₋₄ alkylcarbonyl, or formyl; and further being optionally interrupted by —O—, —N(R^{g})—, —N(R^{g})—C(O)—O—, —O—C(O)—N(R^{g})—, —N(R^{g})—C(O)—N(R^{h})—, —O—C(O)—, —C(O)—O—, or —O—C(O)—O—; each of R^{g} and R^{h}, independently, being hydrogen, alkyl, alkenyl, alkynyl, alkoxy, hydroxylalkyl, hydroxyl, or haloalkyl, wherein the carbon bonded to Y² is unsaturated, and provided that when L is a C₄₋₅ hydrocarbon chain and contains two double bonds, Y¹ is not CH₂;
R¹ is hydrogen, alkyl, alkenyl, alkynyl, alkoxy, hydroxylalkyl, hydroxyl, haloalkyl, or an amino protecting group; and
R² is hydrogen, alkyl, hydroxylalkyl, haloalkyl, or a hydroxyl protecting group;
or a pharmaceutically acceptable salt thereof.

58. The method of claim 57, wherein the compound is 7-phenyl-2,4,6-heptatrienoylhydroxamic acid.

59. A method of inducing apoptosis in a cell comprising contacting the cell with an effective amount of 7-phenyl-2,4,6-heptatrienoylhydroxamic acid, or a pharmaceutically acceptable salt thereof.

60. A method of inducing cell cycle arrest in a cell comprising contacting the cell with an effective amount of 7-phenyl-2,4,6-heptatrienoylhydroxamic acid, or a pharmaceutically acceptable salt thereof.

61. A method of inhibiting the deacetylation of p53 in a cell comprising contacting the cell with an effective amount of 7-phenyl-2,4,6-heptatrienoylhydroxamic acid, or a pharmaceutically acceptable salt thereof.

62. A method of increasing levels of p21 in a cell comprising contacting the cell with an effective amount of 7-phenyl-2,4,6-heptatrienoylhydroxamic acid, or a pharmaceutically acceptable salt thereof.

63. A method of treating hormone-refractory metastatic prostate cancer in a mammal comprising administering to the mammal an effective amount of suberanilo hydoxamic acid, or a pharmaceutically acceptable salt thereof.
